(19) United States
(12) Patent Application Publication
(10) Pub. No.: US 2004/0029107 A1
(43) Pub. Date: Feb. 12, 2004

(54) Mutations of cryptic splice sites in cre and cre fused proteins for improvement of expression and inducibility

(76) Inventors: Frank Oliver Stefan Edenhofer, Erfstadt (DE); Thomas Wunderlich, Koln (DE); Klaus Rajewsky, Koln (DE)

Correspondence Address: WILEY, REIN & FIELDING, LLP, ATTN: PATENT ADMINISTRATION, 1776 K. STREET N.W., WASHINGTON, DC, 20006, US

(21) Appl. No.: 10/332,176

(22) PCT Filed: Jul. 6, 2001
(86) PCT No.: PCT/EP01/07729

(51) Int. Cl. C12Q 1/70; C07H 21/04; C12N 7/00; C12N 15/86

(52) U.S. Cl. 435/5; 435/456; 435/235.1; 536/23.72

(57) ABSTRACT

The invention relates to a DNA sequence, which codes for a mutant of the bacteriophage P1 recombinase, in which a particular cryptic splicing site is disabled by means of a base mutation which alters the Cre protein sequence. The invention further relates to a DNA sequence which codes for a fusion protein from the named Cre mutants and a ligand-binding domain of a receptor protein. The invention furthermore relates to vectors, micro-organisms and transgenic organisms, containing such a DNA sequence and Cre mutants or Cre fusion proteins which are coded by said DNA sequence.

The present invention relates to a DNA sequence coding for a mutant of bacteriophage P1 recombinase Cre in which a special cryptic splicing site has been eliminated by a base mutation changing the Cre protein sequence, and to a DNA sequence coding for a fusion protein derived from said Cre mutant and a ligand-binding domain of a receptor protein. The invention further relates to vectors, microorganisms and transgenic organisms which contain such a DNA sequence, and to Cre mutants or Cre fusion proteins encoded by said DNA sequences.

### BACKGROUND OF THE INVENTION

Well-aimed alterations in the genome of mammals can be effected by means of site-specific recombinases. The bacteriophage P1 recombinase Cre recognizes specific base sequences (loxP sites) and is capable, depending on the position and combination of these loxP sites in the genome, of deleting, inverting, translocating or otherwise desirably mutating genes (for a survey, see Rajewsky et al., J. Clin. Invest. 98, 51-53 (1996)). For inducing the mutation event at any time desired in a selected tissue or cell type of mice, posttranslational switches for Cre activity have been developed, inter alia (Schwenk et al., Nucleic Acids Research 26, 1427-1432 (1998)). These are fusion proteins derived from Cre and the ligand-binding domain of steroid receptors (hereinafter referred to as “LBD”, for example, progesterone receptor, estrogen receptor or glucocorticoid receptor). Such Cre-LBD fusion proteins are inactive with respect to their Cre recombinase functionality, but can be activated by the addition of the corresponding ligands (posttranslationally). In cell cultures and transgenic animals, mutants of LBD were tested which prefer synthetic ligands over natural ones in order that Cre activation may not be triggered by endogenous hormones.

The application of this technology in transgenic mice using both ubiquitously active and cell-type specific promoters has been published (e.g., Schwenk et al. (1998); Kellendonk et al., J. Mol. Biol. 285, 175-182 (1999)). Although it is shown in these works that posttranslational activation of Cre through LBD functions in principle, the system has two critical disadvantages:

1) a reduced activity of Cre-LBD as compared to non-fused Cre;

2) Background activity of Cre-LBD, i.e., Cre recombinase activity even in the absence of the ligand.

For applications in vivo, the background activity has proven problematic, especially when this event occurs rather early in the course of development. This produces mosaic animals in which the recombination event has already occurred in part of the cells while posttranslational control is still possible in the remaining cells. It would be desirable to have a better controlled Cre regulation system which has no (measurable) background activity. This is where the present invention comes into play.

Possible causes of the background activity include:

1) cryptic splicing of the Cre-LBD-encoding mRNA in such a way that a truncated residual protein having non-regulated Cre activity can be translated; and

2) posttranslational degradation, e.g., by endogenous proteases which process an unregulated Cre.

### SUMMARY OF THE INVENTION

Surprisingly, it has been found that these undesirable processes are eliminated when specific modifications are introduced into Cre-coding sequences, such as Cre-LBD-coding sequences:

1) Optimization of the Cre-coding sequence with respect to the exclusion of critical splicing events;

2) mutation or shortening of the linker region between Cre and LBD to render the accession of proteases to this region more difficult.

Thus, the present invention relates to:

(1) a DNA sequence coding for a mutant of bacteriophage P1 recombinase Cre in which the cryptic splicing site in the sequence ATG GTG CGC, which corresponds to positions 1003-1011 in the wild-type sequence shown in SEQ ID NO: 1, has been eliminated by a base mutation changing the Cre protein sequence;

(2) a DNA sequence coding for a fusion protein derived from bacteriophage P1 recombinase Cre and another functional protein, wherein the sequence encoding bacteriophage P1 recombinase Cre is defined as under (1);

(3) a preferred embodiment of the DNA sequence as defined under (2) wherein said further functional protein is a ligand-binding domain of a receptor protein;

(4) a vector containing a DNA sequence according to (1), (2) and/or (3);

(5) a microorganism or a transgenic organism containing a vector as defined in (4) and/or a DNA sequence as defined in (1), (2) and/or (3);

(6) a Cre mutant or Cre fusion protein encoded by the DNA sequence according to (1), (2) and/or (3);

(7) a process for preparing a Cre mutant or Cre fusion protein as defined under (6), comprising the culturing of a microorganism or a cell structure which has been transformed or transfected with a vector according to (4); and

(8) the use of a DNA sequence according to (2) or (3) for the mutagenesis and/or recombination of target sequences containing loxP sites in organisms.

The present invention will now be described in more detail by the attached Figures and Examples.

### DESCRIPTION OF THE FIGURES

FIG. 1: Splicing pattern of selected Cre-LBD constructs

A to D) Represented are the pre-mRNAs of selected Cre-LBD constructs with cryptic splicing donors (SD) and acceptors (SA). The values stated in parentheses express relative probabilities of the utilization of these sites (“scores”). The score is a measure of how similar the splicing sites are to the consensus sequences (E). 100% correspondence with SA yields a score of 14.2, and the mean value for constitutive exons is 7.9. 100% correspondence with SD yields a score of 12.6, and the mean value for constitutive exons is 8.1. Only those SD and SA whose score is above 4 have been indicated. The scores are calculated by means of the computer program “splice site score calculation” (http://www2.1mcb.osakau.ac.jp/splice/score.html). Abbreviations used: SS, synthetic splicing substrate (Choi et al., Mol. Cell. Biol., 11, 3070-3074 (1991)); Crel9, Cre starting with amino acid 19; hCre2, humanized Cre starting with amino acid 2; PR and ER, LBD of the progesterone or estrogen receptor, respectively, the numbers denoting the amino acid position; ERT2, LBD of estrogen receptor with the amino acid substitutions G400V, M543A, L544A (Feil et al., Biochem. Biophys. Res. Commun. 237, 752-757 (1997)); bpA, polyadenylation signal of the bovine growth hormone; Y, either C or T.

FIG. 2: A cryptic splicing event of a P1CrePR650-914-encoding construct detected by RT-PCR.

Represented is the fusion region of Crel9PR650-914.

A) Sequence comparison of the cryptically spliced sequence (top) with the corresponding unspliced sequence (bottom).

B) Schematic representation of the cryptic splicing event. Important amino acids in this region are indicated. The arrows indicate the course of PCR: After the RT reaction, the sequence was amplified with the 5′-primer and the outer 3′-primer. After isolation from the agarose gel, a semi-nested PCR with the 5′-primer and the inner 3′-primer was effected.

FIG. 3: Sequence comparison of the 3′ ends of the various Cre-coding sequences with the consensus for splicing donors.

Section of the 3′ end of the Cre-coding sequence of

A) P1-Cre

B) hCre

C) P1-CreV336A; and

D) hCreV336A.

Shown is that region of the Cre sequence where the amino acid substitution has been introduced. The base sequence around V336 in P1-Cre shows a high homology with the represented consensus of a splicing donor site. The correspondence still increases in hCre, and thus there is a higher risk of cryptic splicing at this site. In the mutated sequences of P1-Cre and hCre, codon 336 (numbering scheme based on the wild-type sequence shown in SEQ ID NO: 1) was changed from GTG (Val) to GCG (Ala); due to this exchange, splicing is no longer possible since the sequence GTN can be considered essential. The base exchange from T to C is at position 1007, based on the base sequence of the wild type Cre (SEQ ID NO: 1, according to Sternberg et al., 1986).

FIG. 4: Activity test of Cre19PR676-914, hCre19PR676-914 and hCre19V336APR676-914 according to Example 6 The bars represent mean values from three different transfections each with standard deviations. White bars represent percent activities of non-mutated Cre19PR676-914 or hCre19PR676-914, and black bars represent mutated hCre19V336APR676-914. The numbers within the bars indicate the inducibility of the corresponding construct, which is calculated by dividing the percent activity in the induced state by the background. “+”: 100 nM RU486 in the medium; “−”: no inducer in the medium.

FIG. 5: Activity test of mutated P1-Cre as compared with mutated hCre in fusion with PR

The experiment was performed as described in Example 6. Represented are two independent transfection runs whose bars represent mean values from three different transfections each with standard deviations. White bars represent percent activities of the Cre19-LBD constructs relative to CMV-Cre, gray bars represent Cre19V336APR676-914, Black bars represent hCre19V336A-LBD constructs. “+”: 100 nM RU486 in the medium; “−”: no inducer in the medium.

FIG. 6: Schematic comparison of the cryptic splicing patterns of the Cre sequences employed and effects on Cre activity in the absence of the inducer.

The stated percentages refer to a comparison of the represented Cre constructs in fusion with PR676-914 relative to non-fused Cre (=100%).

FIG. 7: Comparing analysis of the inducible Cre activities of Cre*PR on which this invention is based (i.e., hCreV336A-PR650-914) with the previously published CrePR1 (Kellendonk et al., Nucl. Acid Res. 24, 1404-1411 (1996)). A) The experiment was performed as described in Example 6. Represented is the mean value of three different transfections with standard deviations. B) Dose-effect analysis: Reporter cells were transfected with the stated constructs and cultured in the presence of different concentrations of RU486.

### DETAILED DESCRIPTION OF THE INVENTION

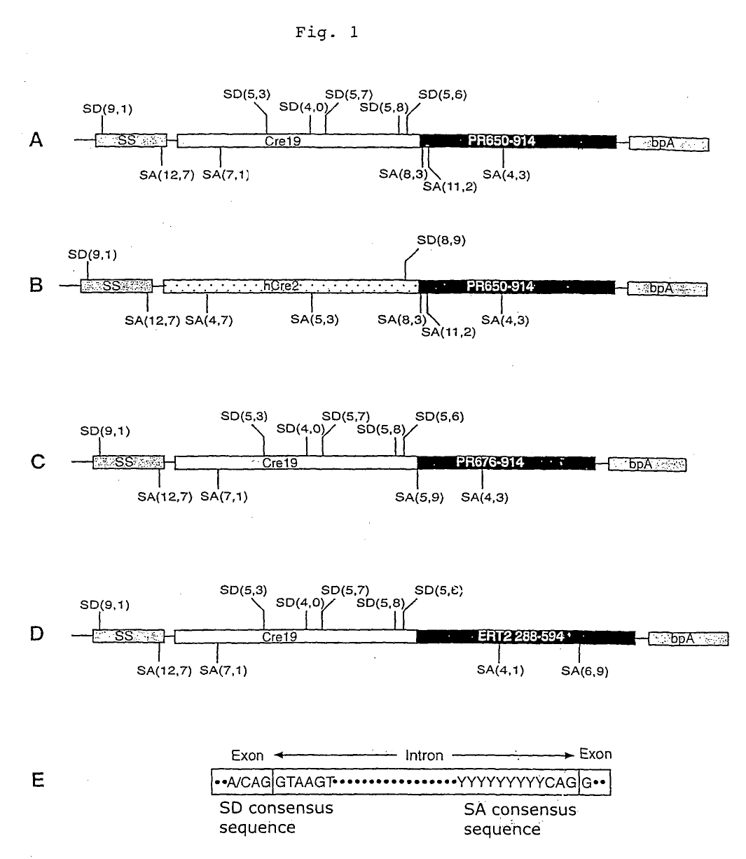

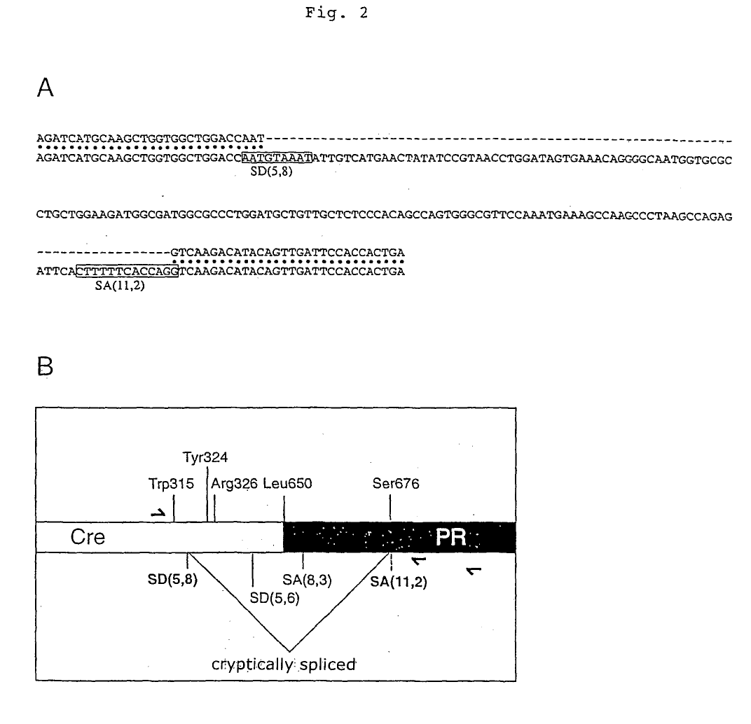

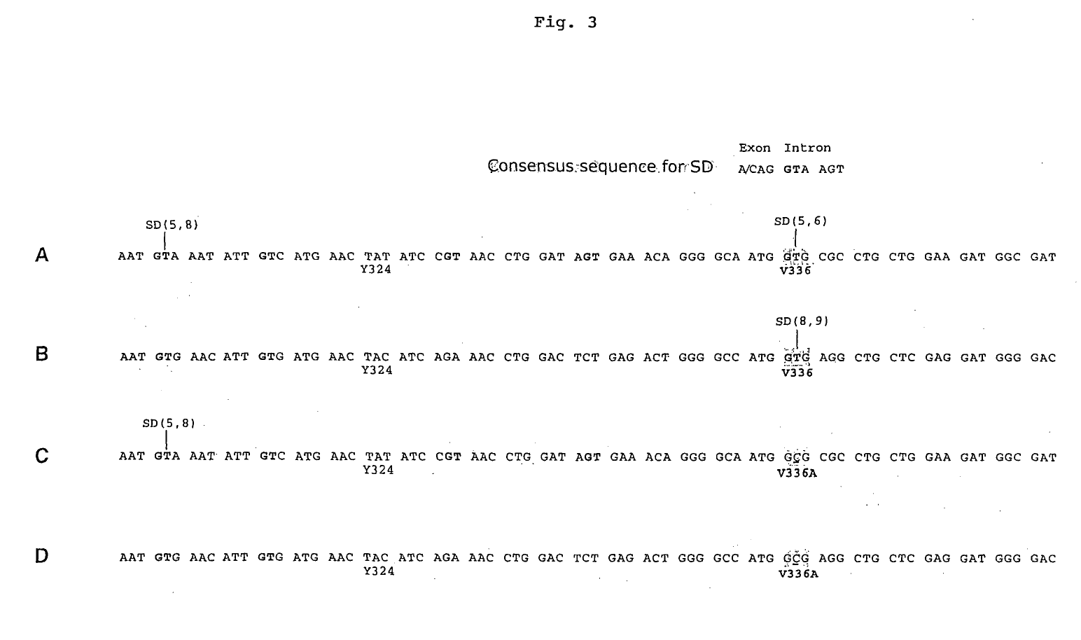

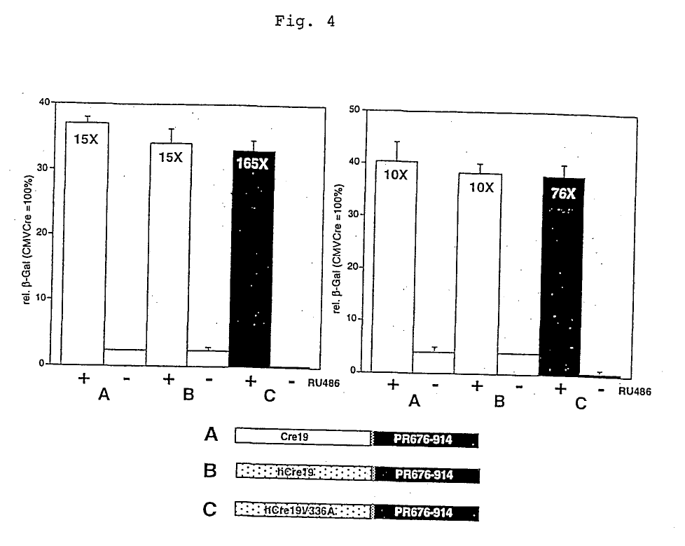

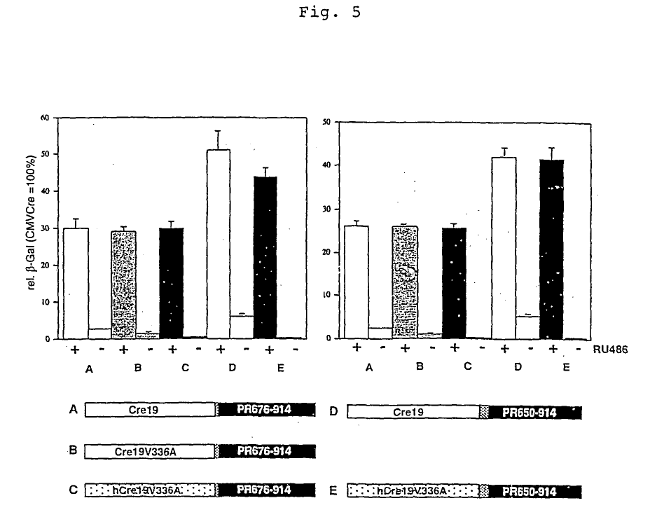

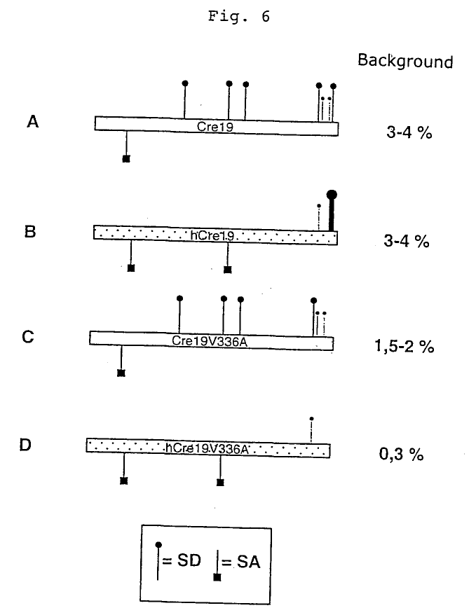

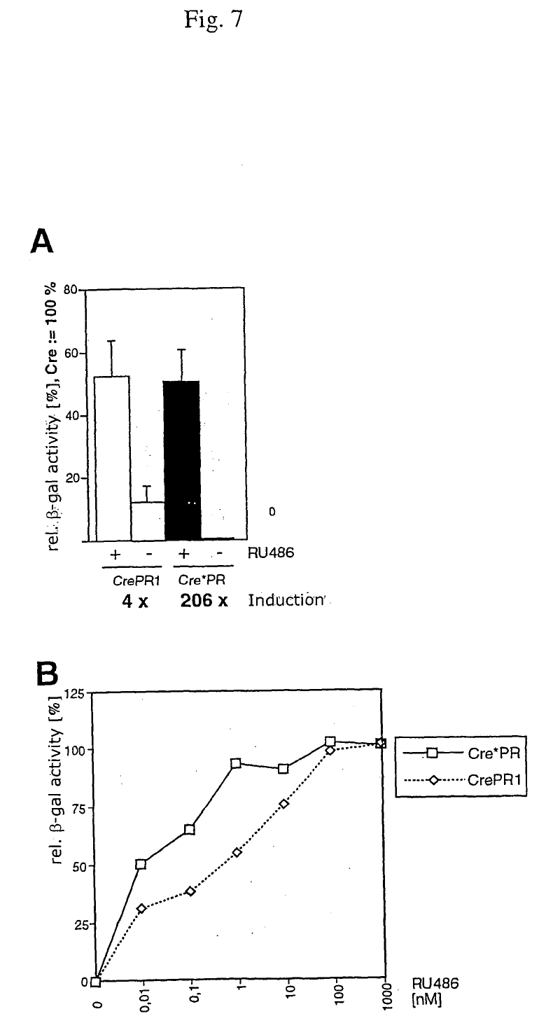

To be able to estimate the extent of cryptic splicing processes in Cre fusion proteins, patterns of potential splicing sites were established by computer analyses (FIG. 1). Within the Cre-coding sequence of bacteriophage P1 (briefly referred to as P1 hereinafter), there appears a relatively strong acceptor at the 5′ terminus as well as five donors of medium strength in the 3′ half (FIG. 1A). In the LBD-coding sequence of progesterone receptor (nucleotides 1948 to 2742 of SEQ ID NO: 3 which code for amino acids 650 to 914 of the wild type receptor, “PR” hereinafter), two acceptors of medium and one SA with extremely high strength appear (FIGS. 1A-B). In the corresponding estrogen-receptor sequence (“ER” hereinafter), the analysis yielded only two SAs of medium strength (FIG. 1D). One or more of the donor sequences positioned at the 3′ end of P1Cre could form an intron with one of the acceptors in PR or ER. By splicing this intron, a premature stop of translation would be generated due to a shift in the reading frame, resulting in a truncated and possibly constitutively active Cre protein without the regulatory portion of LBD. By RT-PCR, it could be proven that such cryptic splicing actually occurs in eukaryotic cells (see FIG. 2). The functionality of the SAs (11.2) in the PR in a physiological context has been published by Balleine et al., J. Clin. End. Meta. 84, 1370-1377 (1999)

In this publication, an alternatively spliced transcript of human progesterone receptor is described which occurs accumulated in breast cancer tumors. Normally, the cryptic splicing of a very low proportion of transgenic transcripts is of less importance. However, in the case of Cre-LBD transcripts, even a minute amount of cryptic splicing products can account for the occurrence of the Cre background activity observed.

Although the Cre sequence (F. Stewart, unpublished results) is improved in this respect by an assimilation of the codon usage since many cryptic splicing sites are eliminated by silent mutations (FIGS. 1A and B), a splicing donor site directly at the 3′ end will not be eliminated, but to the contrary, it is rather enhanced by a codon optimization (“SD (8,9)” in FIG. 1B). When the base sequence in this position is observed (FIG. 3), it becomes evident that elimination of this SD by a silent mutation is not possible: each codon coding for valine (GTN) at the amino acid position 336 of the wild type includes the two bases GT which are critical to the splicing event. A mutation has been introduced which destroys the critical GT sequence (GTG to GCG, exchange on base position 1007, based on the coding base sequence of the wild type Cre sequence, cf. Sternberg et al., 1986), putting up with a conservative amino acid exchange from valine to alanine. This mutation was introduced in both P1-Cre (FIG. 3C) and hcre (FIG. 3D); thus, in hCreV336A, all cryptic SD sites at the 3′ end have been eliminated (FIG. 3D), while in P1-CreV336A, one SD is still present (FIG. 3C).

Using a reporter cell line containing loxP-stop-loxP-lacZ (Kellendonk et al., 1999), the constructs were quantitatively examined for their inducibility, especially Cre activity, in the absence of an inducer (FIG. 4). The hCre-PR-coding construct optimized by the V336A mutation actually shows a background which almost can no longer be measured (FIG. 4C) and is lower by about a factor of 10 as compared with the non-mutated construct (FIG. 4B). Accordingly, the inducibility is improved from about ten- to fifteenfold to from 76- to 165-fold. The introduction of the mutation V336A into the P1-Cre sequence results in a background activity (FIG. 5B) which is between that of the mutated hcre construct (FIG. 5C) and that of non-mutated P1-Cre (FIG. 5A). This could be attributable to the further cryptic splicing sites contained in this construct based on P1-Cre (cf. FIG. 6C). The maximum decrease of the background is due to the construct having the fewest cryptic donor sites, namely hCreV336A-PR (cf. FIG. 6D).

Another improvement could be achieved with respect to maximum activity. The Cre-PR fusion proteins represented in FIG. 4 exhibit a maximum activity of only from 30 to 40% as compared with constitutively active non-fused Cre. From former studies in our laboratory, it was known that other variants of Cre-PR (e.g., Cre-PR650-914) exhibit a clearly higher activity of up to above 500/% of that of Cre. However, these constructs always also have a very high background (see FIG. 5D), which is why they are hardly suitable for use in a transgenic animal. By introducing the above described mutation V336A into this CrePR variant, the undesirable background can be reduced to a level which is again almost not measurable while a high activity of above 40% remains (FIG. 5E).

The construct hCreV336A-PR650-914 on which this invention is based (referred to as Cre*PR hereinafter) was compared in a direct comparison with a previously published and also optimized construct, CrePR1 (Kellendonk et al., Nucl. Acid Res. 24, 1404-1411 (1996)), under identical experimental conditions. Thus, Cre*PR exhibits a 50-fold reduced background activity as compared with CrePR1, combined with a very high increase in inducibility (FIG. 7A). In addition, dose-effect analyses have shown that Cre*PR responds to significantly lower concentrations of RU486 as compared to the previously employed CrePR1 (FIG. 7B).

In the DNA sequence according to the invention (as defined above under (1)), the base mutation changing the Cre protein sequence and eliminating the cryptic splicing site in the Cre DNA sequence is preferably a base substitution in which the codon GTG in the cryptic splicing site sequence defined above under (1) has been replaced by a codon XYZ in which X, Y and Z are independently the nucleotides A, T, C or G, provided that if X=G, then Y≠T (i.e., if Y=T, then X≠G). This means that any sequence which in this position contains the sequence GT which is not critical to the splicing process is suitable. In particular, those base sequences are preferred which result in a conservative amino acid exchange in the resulting protein sequence. Since the sequence GTG codes for Val, this means that sequences coding for aliphatic amino acids, such as alanine (XYZ is GCN), leucine (XYZ is CTN, TTA, TTG) and Ile (XYZ is ATA, ATC or ATT), are particularly preferred. Preferred DNA sequences comprise the partial sequences shown in SEQ ID NOS: 13 and 14.

In addition, in the DNA sequence according to the invention, other cryptic splicing sites present in the wild type sequence may also be eliminated by silent mutations and/or by base mutations changing the Cre protein sequence. Suitable positions for such mutations can be seen from FIGS. 1 and 6.

In a preferred embodiment of the present invention, the DNA sequence coding for the Cre mutant is also truncated at the 5′ end with respect to the Cre wild type sequence. Any truncation is suitable which does not affect the functionality of the resulting Cre proteins. However, it is preferred that the nucleotides corresponding to positions 1 to 54, preferably 1 to 15, of the wild type are truncated.

In another preferred embodiment of the present invention, the DNA sequence coding for the Cre mutant is also modified at the 5′ end. Within the meaning of the present invention, “modified” means that additional nucleobases may be added both to the 5′ end of the untruncated sequence and to the 5′ end of the Cre DNA sequence truncated as defined above. These additional nucleobases are codons coding for amino acids which are supposed to have a stabilizing effect on the total protein according to the so-called N-end rule (Levy, F. et al., Proc. Natl. Acad. Sci. USA 93, 4907-4912 (1996)). Such stabilizing amino acid sequences essentially consist of neutral amino acids, preferably Met, Gly, Val and Ala. Both sequences which contain combinations of these amino acids as well as homogeneous sequences of one of the amino acids mentioned are possible. The length of the stabilizing amino acid sequences is preferably from 1 to 10 amino acids (corresponding to from 3 to 30 nucleobases), especially from 1 to 3 amino acids. In a particularly preferred embodiment, the DNA sequence contains the sequence ATG GGC GCC (coding for Met-Gly-Ala) starting immediately at the translation origin.

In a preferred embodiment, the DNA sequence according to the invention comprises the nucleobases 1 to 984 of SEQ ID NOS: 5, 7 or 9.

In another preferred embodiment, the DNA sequence is truncated at the 3′ terminus with respect to the wild type sequence. According to the present invention, from 1 to 24 nucleotides, based on the wild type sequence shown in SEQ ID NO: 1, may be truncated (i.e., truncation behind the mutation changing the Cre protein sequence which is present in position 1006-1008). Deletions of single codons downstream from the codon 1006-1008 are also possible according to the present invention.

In the DNA sequence coding for a fusion protein, the functional protein is selected from the group of proteins consisting of enzymes, peptide hormones, pharmacologically active peptides and structural proteins. The DNA sequences which code for the functional proteins may be positioned both downstream or upstream from the DNA sequence coding for the Cre mutant. However, the stabilizing effect of the Cre mutant sequence according to the invention is manifested especially if the DNA sequence coding for the functional protein is downstream from the Cre DNA sequence, i.e., is linked to the 3′ end of the Cre DNA sequence.

Preferably, the functional protein is the ligand-binding domain of a receptor protein, especially a ligand-binding domain of a steroid receptor, such as progesterone, estrogen or glucocorticoid receptor. These DNA sequences can be derived from any mammals, preferably from humans, mice or rats. More preferred is a ligand-binding domain of a steroid receptor, especially one comprising the nucleotides 1948 to 2742 of the human sequence shown in SEQ ID NO: 3.

The above defined preferred DNA sequences coding for fusion proteins may contain further fusion partners (DNA sequences coding for functional proteins), e.g., those which enable easier purification (such as glutathione-S-transferase, GST, or maltose-binding protein, MBP), detection (e.g., green fluorescent protein, GFP) or transduction (e.g., transduction domains of HIV-TAT or VP22). In addition, the present invention also relates to DNA sequences coding for fusion proteins which comprise the Cre sequence according to the invention and the further fusion partners mentioned.

Further, it is preferred that the linker sequence in the fusion protein between the Cre domain and the DNA sequence coding for the functional protein, especially for the ligand-binding domain, be as short as possible and/or mutated to impede the access for proteases. For the DNA sequences coding for these fusion proteins, this means that if the DNA coding for the functional protein (or the ligand-binding domain) is downstream from the Cre DNA sequence, then the region behind the mutated cryptic splicing site (at positions 1006-1008, based on the wild type sequence) to the end of the Cre sequence or to the functional start of the ligand-binding domain as defined above may be wholly or partially deleted and/or mutated. The term “mutated” also includes the case that the original DNA sequence is replaced by a short DNA sequence which codes for an amino acid sequence which essentially consists of neutral amino acids, such as Gly, Ala, Val, Pro or Ile, especially Gly and Pro. In particular, the length of this amino acid sequence is within a range of from 1 to 10, preferably from 1 to 5, amino acids.

Preferably, the splicing sites occurring within the sequence coding for the ligand-binding domain have also been eliminated by silent mutation or by a base mutation changing the protein sequence of the ligand-binding domain. More preferred sequences are the DNA sequences shown in SEQ ID NOS: 5, 7 and 9.

The vector according to the invention as defined above under (3) may contain additional functional sequences, such as the sequences defined above which enable purification, detection or transduction, in addition to the DNA sequence (1) and/or (2). Further, it may also contain promoter sequences which enable expression, e.g., in bacteria or yeasts or lepidoptera cells, or expression in transgenic organisms as defined above. In addition, the vector may include replication origins, selectable markers and multiple cloning sites and the like.

The microorganisms or transgenic organisms according to the invention may both be transformed with the vector according to the invention as defined above and have the DNA sequence according to the invention integrated in their chromosomes. “Microorganisms” according to the present invention includes both prokaryotes and eukaryotes. “Organisms” according to the present invention includes vertebrates, especially mammals (including human and non-human mammals, more especially rodents such as mice or rats) or fishes, or invertebrates, such as worms and flies.

In addition to the above defined sequences, the Cre mutant according to the invention or the Cre fusion protein according to the invention may also contain other proteinogenic structures or be linked to non-proteinogenic structures.

The process according to the invention for preparing the Cre mutant or the Cre fusion protein may be effected, as defined above, by culturing a microorganism transformed with a suitable vector. The target proteins may then be isolated and purified from the culture by methods known to the skilled person.

The invention is of great importance to experiments (both in vivo and in vitro) which relate to the conditional expression of genes. The sequence according to the invention is useful, in particular, if no background activity of the induction is to be tolerated. For example, upon application of a former Cre-LBD system, it was reported that the inducible expression of a reporter gene was limited by spontaneous expression. This background activation is explained by a ligand-independent recombinase activity of Cre-LBD (Fuhrmann-Benzakein et al., Nucl. Acids Res. 28, e99 (2000)). The application of a construct according to the invention can overcome such limitation. The generation and analysis of Cre*PR-transgenic mice is currently under research.

The invention is further of particular importance to the further development of inducible conditional gene targeting. “Conditional gene targeting” means the selective mutagenesis of a gene in a freely selectable cell type or tissue; also, the mutagenesis may optionally be effected at any time desired using an inducible system. These modifications of classical gene targeting are important especially when the mutation already occurring in germ cells exhibits undesirable side-effects. Using the invention, transgenic animals, preferably mice, can be prepared which express a posttranslationally inducible Cre fusion protein. Upon crossing with a murine line which carries a loxP-containing target sequence, selected changes in the genome, such as deletions, inversions or translocations, can be effected at any time desired by adding an inducer. One particular advantage of the invention resides in the low Cre recombinase activity described which prevails prior to the addition of the inducer. Thus, an undesirable (i.e., occurring prior to the addition of the inducer) modification of the genotype is not to be expected in the transgenic animal. To date, such Cre background activity has rendered it difficult to unambiguously establish the genotype before or after induction. Depending on the specificity of the promoter used in the transgene, the expression of the inducible Cre can be effected either in a cell-type or tissue-specific was or ubiquitously. In the latter case, a transgenic animal is obtained which allows the temporal control over mutagenesis in any type of tissue or cells. A control with respect to time and space is enabled by using cell-type specific promoters. The transgenic constructs can be either used by microinjection for generating transgenic lines, or integrated into selected regions of the genome by homologous recombination.

The present invention will now be explained in more detail by the following Examples.

### EXAMPLES

In the Examples described below, the following primers and templates were used:

Primers:

| | |
|---|---|
| 3Bsicre343 | AAATTCGTACGCATCGCCATCTTCCAGCAGG |
| 3Narcre343 | AAATTGGCGCCATCGCCATCTTCCAGCAGG |
| 3Kascre343 | AATTTGGCGCCGTCCCCATCCTCGAGCAG |
| 3Sfomutcre343 | AAATTGGCGCCATCGCCATCTTCCAGCAGGCG |
| | CGCCATTGCCCC |
| 3Sfomutsshcre343 | AAATTGGCGCCGTCCCCATCCTCGAGCAGCCT |
| | CGCCATGGCCCC |
| 5Bamhcre2 | GGGGGATCCACCATGGGTGCCTCCAACCTGCT |
| | GACTGTG |
| 5Bamhcre19 | TTTAAGGATCCACCATGGGTGCCACGAGTGAT |
| | GAGGTTCGCA |
| 5Bsicre19 | TTTAACGTACGGCACGAGTGATGAGGTTCGCA |
| 5Narcre19 | TTTAAGGCGCCACGAGTGATGAGGTTCGCA |
| Templates: | |
| pGKCrebpA | Institut für Genetik, |
| | UniversitÄt zu Köln. |
| pBIuehCre | Seeburg, MPI für med. |
| | Forschung, Heidelberg, |
| | and Stewart, EMBL, Heidelberg. |
| pNNCre19PR676-914 | Institut für Genetik, |
| | Universität zu Köln. |
| pNNhCre19PR676-914 | Institut für Genetik, |
| | Universität zu Köln. |
| pNN265E-bpA | Artemis, Cologne. |

### Example 1

### Cloning Step I: LBD in pNN

In the first cloning step, the various lengths of the LBD of PR and of ER were cloned into pNN265E-bpA (obtainable from the Institut für Genetik, Univ. zu Köln, Germany). The cloning was effected through the restriction enzymes BamHI and EcoRV which cut only once in the vector and which were also introduced into the amplificates by PCR.

Various lengths of the LBD of PR were generated by 5′- and 3′-terminal truncations. The 5′ truncations began at amino acids 650, 676 and 678 of PR. By beginning the PR-LBD at Leu650, the D domain of the LBD is shortened by 10 amino acids. This N terminus was selected since such constructs have high activities in the context with Cre, as known from former experience (Kellendonk et. al., Nucl. Acid Res. 24, 1404-1411 (1996)). A second N-terminal truncation was effected up to Ser676 of PR. This was based on the result found by Kellendonk et al. (1996) and consideration resulting therefrom: The less amino acids of the D domain of PR there are as linkers between Cre and LBD, the lower the background activity of the fusion proteins. Inter alia, the amino acids 641, 672 and 687 were tested as starting points for LBD (Kellendonk et al., 1996). PR641 showed a high activity in the context with Cre, but also a high background. In contrast, PR672 showed a little less activity and also less background as compared to PR641. PR687 showed hardly any activity at all. Therefore, the starting point of the Ser676 construct was chosen between amino acids 672 and 687, with the expectation that a high activity of Cre is retained and the background also remains on a tolerable low level. In addition, in Ser676 constructs, in contrast to Leu650, two strong splicing acceptors (SA) for possible cryptic splicing have been modified on the sequence level. One SA is eliminated by deleting the sequence. The second SA is changed in its consensus sequence in such a way that the high C/T-rich sequence segment is lacking in this construct. In addition, in the linker region, it has been taken care that possible protease recognition amino acids, such as Arg, Lys, are deleted. In the third 5′ truncation variant Gly678, the remaining sequence of the second SA, which is still present in Ser676, was deleted. In order that the Cre activity in such constructs is not limited any further by shortening the linker, two glycines, which are also intended to enhance the flexibility of the linker, were substituted for Ser 676 and Pro677.

### Example 2

### Cloning of Cre19 to the N or C Terminus of the LBD

The non-directed cloning of Cre19 to the N terminus of the LBD was effected through the KasI restriction site. The insert amplified on the template pGKCrebpA (Table 1) was digested with KasI and ligated into KasI-restricted and then dephosphorylated pNN vectors with LBD. In addition, Cre19 was cloned in a non-directed way to the C-terminal end of the LBD through the BsiWI restriction site of the pNN vectors with the various LBDs of PR and ER. Thus, the Crel9 sequence was amplified by PCR on the template pGKCrebpA with the primers represented in Table 1, restricted with BsiWI and ligated into BsiWI-restricted, dephosphorylated pNN vectors.

TABLE I
Primer sequences employed

| | 5′-Primer | 3′-Primer | Template |
|---|---|---|---|
| N-terminal Cre19 | 5Narcre19 | 3Narcre343 | pGKCrebpA |
| c-terminal Cre19 | 5Bsicre19 | 3Bsicre343 | pGKCreboA |

### Example 3

### Cloning of hCre to the N Terminus of PR676-914

Since Cre is a phage protein and its sequence is therefore adapted to the prokaryotic translation machinery, it was attempted to achieve an optimization for the eukaryotic expression system. R. Sprengel optimized the sequence of Cre accordingly in cooperation with F. Stewart to thus obtain an improved translation efficiency. He called the thus altered Cre sequence “humanized Cre” (hCre) (F. Stewart, unpublished results). After it was found that the construct pNNCre19PR676-914 exhibited the best Cre activities to date in a cell culture, an hCre was inserted in this construct in place of the normal Cre19.

The sequences of hCre2 and hCre19 were respectively cloned to the N terminus of PR676914. PCR of hCre19 and hCre2 was effected on the template pBluehCre with the primers stated in Table 2, the amplificates were restricted with BamHI/KasI and ligated into a BamHI/KasI-restricted pNNCre19PR676-914.

TABLE 2
Primer sequences employed

| | 5′-Primer | 3′-Primer | Template |
|---|---|---|---|
| N-terminal hCre2 | 5Bamhcre2 | 3Kascre343 | pBIuehCre |
| N-terminal hCre19 | 5Bamhcre19 | 3Kascre343 | pBluehCre |

### Example 4

### Cloning of Cre19V336A

The mutation V336A was introduced into the phage Cre19 sequence. Thus, the Cre sequence comprising mutation, referred to as Cre19V336A in the following, was clone to the PR676-914 sequence.

The mutation V336A was introduced into the Cre19 sequence through the 3′ primer stated in Table 3. The amplificate was amplified on the template pNNCre19PR676-914 and cloned in a non-directed way through its SfoI restriction sites into an SfoI-restricted and subsequently dephosphorylated pNNCre19PR676914. By digestion with BamHI, the right orientation of Cre19V336A within the vector could be shown. The mutation was detected by sequencing.

TABLE 3
Primer sequences employed

| | 5′-Primer | 3′-Primer | Template |
|---|---|---|---|
| Cre19V336A | 5Narcre19 | 3Sfomutcre343 | pNNCre19PR676-914 |

### Example 5

### Cloning of hCre19V336A

The mutation V336A was introduced into the sequence of hCre19 through the 3′-primer stated in Table 4. Through the BamHI and SfoI cloning sites introduced in the template pNNhCre19PR676-914 by PCR, hCre19V336A was cloned into a BamHI/SfoI-restricted pNNCre19PR676-914. The mutation was detected by sequencing.

TABLE 4

| | 5′-Primer | 13′-Primer | Template |
|---|---|---|---|
| hCre19V336A | 5Bamhhcre19 | 3Sfomutsshcre343 | pNNhCre19- |
| | | | PR676-914 |

### Example 6

### Activity test on Cre19-PR676-914, hCre19-PR676-914 and hCre19V336A-PR676-914

CV1-5B cells sown on the previous day into wells of 6-well plates (8×10⁴ cells/6well/transfection) were transfected in triplicate with 1 μg of the respective fusion protein plasmids and 1 μg of pHD2-AP. Twelve hours after the transfection, the cells were trypsinated off and divided in equal portions to 2 wells of a 12-well plate. To one well was added 100 nM RU486 (obtainable from Sigma Chemie GmbH, Deisenhofen, Germany) in medium, and empty medium was added to the other. Three days after the transfection, the cells were fixed and stained against β-galactosidase and alkaline phosphatase (for normalization with respect to transfection efficiency). The percent comparison was effected against pNN-CMV-Cre transfected in parallel (Cre without a fusion partner in the same vector). Thus, the mean value of CMV-Cre was set to 100%, and the mean values for the fusion proteins were compared with this. The result is summarized in FIG. 4.

1. A DNA sequence coding for a mutant of bacteriophage P1 recombinase Cre in which the cryptic splicing site in the sequence ATG GTG CGC, which corresponds to positions 1003-1011 in the wild-type sequence shown in SEQ ID NO: 1, has been eliminated by a base mutation changing the Cre protein sequence.

2. The DNA sequence according to claim 1, wherein the codon GTG in said splicing site sequence has been replaced by a codon XYZ, in which X, Y and Z are independently the nucleotides A, T, C or G, provided that if X=G, then Y≠T.

3. The DNA sequence according to claim 2, wherein codon XYZ codes for a neutral amino acid and, in particular, is the Ala-coding codon GCG.

4. The DNA sequence according to one or more of claims 1 to 3, wherein other cryptic splicing sites present in the wild type sequence have been eliminated by silent mutation and/or by base mutations changing the Cre protein sequence.

5. The DNA sequence according to any of claims 1 to 4, which is truncated at the 5′ terminus with respect to the Cre wild type sequence, especially a DNA sequence in which the nucleotides corresponding to positions 1 to 54 of the wild type are truncated.

6. The DNA sequence according to any of claims 1 to 5, wherein the DNA sequence has nucleotides at the 5′ end which code for additional amino acids not present in the wild type Cre sequence.

7. The DNA sequence according to claim 6, wherein said additional nucleotides code for neutral amino acids, especially Met, Gly, Val and Ala.

8. The DNA sequence according to one or more of claims 1 to 7, wherein said DNA sequence is truncated at the 3′ terminus with respect to the wild type sequence.

9. The DNA sequence according to claim 1, wherein said DNA sequence comprises the nucleotides 1 to 984 of SEQ ID NOS: 5, 7 or 9.

10. A DNA sequence coding for a fusion protein derived from bacteriophage P1 recombinase Cre and another functional protein, wherein the sequence encoding bacteriophage P1 recombinase Cre is defined as in claims 1 to 9.

11. The DNA sequence according to claim 10, wherein said functional protein is a ligand-binding domain of a receptor protein, the receptor being, in particular, a steroid receptor, preferably a progesterone, estrogen or glucocorticoid receptor.

12. The DNA sequence according to claim 11, wherein said receptor is a progesterone receptor, especially a progesterone receptor which comprises the nucleotides 1948 to 2742 of SEQ ID NO: 3.

13. The DNA sequence according to any of claims 10 to 12, wherein cryptic splicing sites within the sequence coding for the ligand-binding domain have been eliminated by silent mutation or by a base mutation changing the protein sequence of said ligand-binding domain.

14. The DNA sequence according to claim 10, having the sequence shown in SEQ ID NOS: 5, 7 or 9.

15. A vector containing a DNA sequence according to one or more of claims 1 to 14.

16. A microorganism or transgenic organism containing a vector as defined in claim 15 and/or a DNA sequence as defined in claims 1 to 14.

17. A Cre mutant or Cre fusion protein encoded by the DNA sequence according to claims 1 to 14.

18. A process for preparing a Cre mutant or Cre fusion protein as defined in claim 17, comprising the culturing of a microorganism or a cell culture which has been transformed or transfected with a vector according to claim 15.

19. Use of a DNA sequence according to claims 11 to 14 for the mutagenesis and/or recombination of target sequences containing loxP sites in organisms.


SEQUENCE LISTING (24 sequences)

SEQ ID NO: 1 - DNA, 1032 bp, Bacteriophage P1
  CDS (1)..(1029)
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 2 - protein, 343 aa, Bacteriophage P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 3 - DNA, 2802 bp, Homo sapiens
  CDS (1)..(2799)
atgactgagctgaaggcaaagggtccccgggctccccacgtggcgggcggcccgccctcccccgaggtcggatccccactgctgtgtcgcccagccgcaggtccgttcccggggagccagacctcggacaccttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcggactgctggacagtgtcttggacactctgttggcgccctcaggtcccgggcagagccaacccagccctcccgcctgcgaggtcaccagctcttggtgcctgtttggccccgaacttcccgaagatccaccggctgcccccgccacccagcgggtgttgtccccgctcatgagccggtccgggtgcaaggttggagacagctccgggacggcagctgcccataaagtgctgccccggggcctgtcaccagcccggcagctgctgctcccggcctctgagagccctcactggtccggggccccagtgaagccgtctccgcaggccgctgcggtggaggttgaggaggaggatggctctgagtccgaggagtctgcgggtccgcttctgaagggcaaacctcgggctctgggtggcgcggcggctggaggaggagccgcggctgtcccgccgggggcggcagcaggaggcgtcgccctggtccccaaggaagattcccgcttctcagcgcccagggtcgccctggtggagcaggacgcgccgatggcgcccgggcgctccccgctggccaccacggtgatggatttcatccacgtgcctatcctgcctctcaatcacgccttattggcagcccgcactcggcagctgctggaagacgaaagttacgacggcggggccggggctgccagcgcctttgccccgccgcggagttcaccctgtgcctcgtccaccccggtcgctgtaggcgacttccccgactgcgcgtacccgcccgacgccgagcccaaggacgacgcgtaccctctctatagcgacttccagccgcccgctctaaagataaaggaggaggaggaaggcgcggaggcctccgcgcgctccccgcgttcctaccttgtggccggtgccaaccccgcagccttcccggatttcccgttggggccaccgcccccgctgccgccgcgagcgaccccatccagacccggggaagcggcggtgacggccgcacccgccagtgcctcagtctcgtctgcgtcctcctcggggtcgaccctggagtgcatcctgtacaaagcggagggcgcgccgccccagcagggcccgttcgcgccgccgccctgcaaggcgccgggcgcgagcggctgcctgctcccgcgggacggcctgccctccacctccgcctctgccgccgccgccggggcggcccccgcgctctaccctgcactcggcctcaacgggctcccgcagctcggctaccaggccgccgtgctcaaggagggcctgccgcaggtctacccgccctatctcaactacctgaggccggattcagaagccagccagagcccacaatacagcttcgagtcattacctcagaagatttgtttaatctgtggggatgaagcatcaggctgtcattatggtgtccttacctgtgggagctgtaaggtcttctttaagagggcaatggaagggcagcacaactacttatgtgctggaagaaatgactgcatcgttgataaaatccgcagaaaaaactgcccagcatgtcgccttagaaagtgctgtcaggctggcatggtccttggaggtcgaaaatttaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgcacaattacccaagatattggcagggatggtgaaaccccttctctttcataaaaagtga

SEQ ID NO: 4 - protein, 933 aa, Homo sapiens
MTELKAKGPRAPHVAGGPPSPEVGSPLLCRPAAGPFPGSQTSDTLPEVSAIPISLDGLLFPRPCQGQDPSDEKTQDQQSLSDVEGAYSRAEATRGAGGSSSSPPEKDSGLLDSVLDTLLAPSGPGQSQPSPPACEVTSSWCLFGPELPEDPPAAPATQRVLSPLMSRSGCKVGDSSGTAAAHKVLPRGLSPARQLLLPASESPHWSGAPVKPSPQAAAVEVEEEDGSESEESAGPLLKGKPRALGGAAAGGGAAAVPPGAAAGGVALVPKEDSRFSAPRVALVEQDAPMAPGRSPLATTVMDFIHVPILPLNHALLAARTRQLLEDESYDGGAGAASAFAPPRSSPCASSTPVAVGDFPDCAYPPDAEPKDDAYPLYSDFQPPALKIKEEEEGAEASARSPRSYLVAGANPAAFPDFPLGPPPPLPPRATPSRPGEAAVTAAPASASVSSASSSGSTLECILYKAEGAPPQQGPFAPPPCKAPGASGCLLPRDGLPSTSASAAAAGAAPALYPALGLNGLPQLGYQAAVLKEGLPQVYPPYLNYLRPDSEASQSPQYSFESLPQKICLICGDEASGCHYGVLTCGSCKVFFKRAMEGQHNYLCAGRNDCIVDKIRRKNCPACRLRKCCQAGMVLGGRKFKKFNKVRVVRALDAVALPQPVGVPNESQALSQRFTFSPGQDIQLIPPLINLLMSIEPDVIYAGHDNTKPDTSSSLLTSLNQLGERQLLSVVKWSKSLPGFRNLHIDDQITLIQYSWMSLMVFGLGWRSYKHVSGQMLYFAPDLILNEQRMKESSFYSLCLTMWQIPQEFVKLQVSQEEFLCMKVLLLLNTIPLEGLRSQTQFEEMRSSYIRELIKAIGLRQKGVVSSSQRFYQLTKLLDNLHDLVKQLHLYCLNTFIQSRALSVEFPEMMSEVIAAQLPKILAGMVKPLLFHKK

SEQ ID NO: 5 - DNA, 1725 bp, Artificial Sequence
  Fusion protein of bacteriophage P1-Cre19V336A- PR676-914
atgggcgccacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggcgcgcctgctggaagatggcgatggcgcctcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctatgcatgcgtacggatag

SEQ ID NO: 6 - protein, 574 aa, Artificial Sequence
  Fusion protein of bacteriophage P1-Cre19V336A- PR676-914
MGATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMARLLEDGDGASPGQDIQLIPPLINLLMSIEPDVIYAGHDNTKPDTSSSLLTSLNQLGERQLLSVVKWSKSLPGFRNLHIDDQITLIQYSWMSLMVFGLGWRSYKHVSGQMLYFAPDLILNEQRMKESSFYSLCLTMWQIPQEFVKLQVSQEEFLCMKVLLLLNTIPLEGLRSQTQFEEMRSSYIRELIKAIGLRQKGVVSSSQRFYQLTKLLDNLHDLVKQLHLYCLNTFIQSRALSVEFPEMMSEVIAMHAYG

SEQ ID NO: 7 - DNA, 1725 bp, Artificial Sequence
  Fusion protein hCre19V336A-PR676-914
atgggtgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggcgaggctgctcgaggatggggacggcgcctcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctatgcatgcgtacggatag

SEQ ID NO: 8 - protein, 574 aa, Artificial Sequence; identical to SEQ ID NO: 6
  Fusion protein hCre19V336A-PR676-914

SEQ ID NO: 9 - DNA, 1800 bp, Artificial Sequence
  Fusion protein hCre19V336A-PR650-914
atgggtgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggcgaggctgctcgaggatggggacggcgccctggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctatgcatgcgtacggatag

SEQ ID NO: 10 - protein, 599 aa, Artificial Sequence
  Fusion protein hCre19V336A-PR650-914
MGATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMARLLEDGDGALDAVALPQPVGVPNESQALSQRFTFSPGQDIQLIPPLINLLMSIEPDVIYAGHDNTKPDTSSSLLTSLNQLGERQLLSVVKWSKSLPGFRNLHIDDQITLIQYSWMSLMVFGLGWRSYKHVSGQMLYFAPDLILNEQRMKESSFYSLCLTMWQIPQEFVKLQVSQEEFLCMKVLLLLNTIPLEGLRSQTQFEEMRSSYIRELIKAIGLRQKGVVSSSQRFYQLTKLLDNLHDLVKQLHLYCLNTFIQSRALSVEFPEMMSEVIAMHAYG

SEQ ID NO: 11 - DNA, 81 bp, Artificial Sequence
  3′-end of the Cre-coding sequence from bacteriophagen P1-Cre
aatgtaaatattgtcatgaactatatccgtaacctggatagtgaaacaggggcaatggtgcgcctgctggaagatggcgat

SEQ ID NO: 12 - DNA, 81 bp, Artificial Sequence
  3′-end of the Cre-coding sequence from hCre
aatgtgaacattgtgatgaactacatcagaaacctggactctgagactggggccatggtgaggctgctcgaggatggggac

SEQ ID NO: 13 - DNA, 81 bp, Artificial Sequence
  3′-end of the Cre-coding sequence from P1- CreV336A
aatgtaaatattgtcatgaactatatccgtaacctggatagtgaaacaggggcaatggcgcgcctgctggaagatggcgat

SEQ ID NO: 14 - DNA, 81 bp, Artificial Sequence
  3′-end of the Cre-coding sequence from hCreV336A
aatgtgaacattgtgatgaactacatcagaaacctggactctgagactggggccatggcgaggctgctcgaggatggggac

SEQ ID NO: 15 - DNA, 31 bp, Artificial Sequence
  Primer
aaattcgtacgcatcgccatcttccagcagg

SEQ ID NO: 16 - DNA, 30 bp, Artificial Sequence
  Primer
aaattggcgccatcgccatcttccagcagg

SEQ ID NO: 17 - DNA, 29 bp, Artificial Sequence
  Primer
aatttggcgccgtccccatcctcgagcag

SEQ ID NO: 18 - DNA, 44 bp, Artificial Sequence
  Primer
aaattggcgccatcgccatcttccagcaggcgcgccattgcccc

SEQ ID NO: 19 - DNA, 44 bp, Artificial Sequence
  Primer
aaattggcgccgtccccatcctcgagcagcctcgccatggcccc

SEQ ID NO: 20 - DNA, 39 bp, Artificial Sequence
  Primer
gggggatccaccatgggtgcctccaacctgctgactgtg

SEQ ID NO: 21 - DNA, 42 bp, Artificial Sequence
  Primer
tttaaggatccaccatgggtgccacgagtgatgaggttcgca

SEQ ID NO: 22 - DNA, 32 bp, Artificial Sequence
  Primer
tttaacgtacggcacgagtgatgaggttcgca

SEQ ID NO: 23 - DNA, 30 bp, Artificial Sequence
  Primer
tttaaggcgccacgagtgatgaggttcgca

SEQ ID NO: 24 - DNA, 1032 bp, Bacteriophage P1
  CDS (1)..(1029)
  misc_feature (335)..(335): The ′Xaa′ at location 335 stands for Lys, Asn, Arg, Ser, Thr, Ile, Met, Glu, Asp, Gly, Ala, Val, Gln, His, Pro, Leu, Tyr, Trp, Cys, or Phe.
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcannnnnnnnnctgctggaagatggcgattag